(19) United States
(12) Patent Application Publication
(10) Pub. No.: US 2008/0262376 A1
(43) Pub. Date: Oct. 23, 2008

(54) WIRELESS SENSOR SYSTEM FOR MONITORING SKIN CONDITION USING THE BODY AS COMMUNICATION CONDUIT

(75) Inventor: Michael Price, Palo Alto, CA (US)

Correspondence Address: D. BOMMI BOMMANNAN, 2251 Grant Road, Suite B, LOS ALTOS, CA, 94024, US

(73) Assignee: PROACTIVE HEALTH DEVICES, INC., Palo Alto, CA (US)

(21) Appl. No.: 12/105,046

(22) Filed: Apr. 17, 2008

Related U.S. Application Data
(60) Provisional application No. 60/912,418, filed on Apr. 17, 2007.
(60) Provisional application No. 61/019,772, filed on Jan. 8, 2008.

(51) Int. Cl. A61B 5/053

(52) U.S. Cl. 600/547

(57) ABSTRACT

Devices and methods for measuring a local skin parameter or the presence or concentration of an analyte present in a biological medium are disclosed. A monitoring system comprising disposable sensor components and a network component for the collection of sensor information and for relaying this information for remote access and analysis is disclosed, where the sensor components and the network component communicate using the wearer as a signal propagation medium.

### CROSS-REFERENCE TO RELATED APPLICATIONS

This application claims priority to U.S. Provisional Patent Application No. 60/912,418 filed on Apr. 17, 2007, and to U.S. Provisional Patent Application No. 61/019,772 filed on Jan. 8, 2008.

### BACKGROUND

1. Field of the Invention

The field of the invention relates to sensor systems, and in particular to devices and methods for monitoring skin condition using sensors configured to use the body as a communication conduit.

2. Description of the Related Art

Sensors for monitoring health conditions have been an active area of research and development. In some applications, cost and performance requirements make current technologies inappropriate. These applications require some combination of disposability, wireless communication, ease of application, and low cost. One such application is the monitoring of residents in long term care where limited resources make quality health care difficult to maintain.

Skin disease is common in long term care and hospitals, often affecting more than 60% of residents. These diseases include dermatitis, rashes, skin tears, skin shears, lesions, and decubitus ulcers. The cost of daily care for people with skin disease is much higher than for people with healthy skin, due to the requirement that medical professionals diagnose, prescribe, and monitor care. Skin disease is entirely preventable, but healthcare facility managers, particularly facilities for eldercare, struggle with high staff turnover, rapidly rising costs, and a chronic shortage of nurses and aides. Current care procedures are labor intensive.

Incontinence is a significant risk factor for skin disease. Most facilities have a “check regularly and change when needed” policy. Modern super absorbent diapers make checking more difficult by producing a “dry feel” when urine is present, often requiring a more intrusive examination. Sometimes checking is cursory due to other demands on staff time and the unpleasant nature of the checking task. A regular checking schedule may also neglect the differing needs of individual residents in these facilities.

Assessment of whether proper care is being administered is based on the presence or absence of visible early stages of skin disease, such as redness or rash. Once a resident's skin health begins to deteriorate, the complexity and costs of care escalate rapidly. Ointments, salves, and antibiotics may need to be employed and the presence of licensed vocational nurses (LVNs) or registered nurses (RNs), in addition to certified nurse assistants (CNAs), may also be required. Apart from the high costs due to the need for experienced professionals, high staff turnover contributes to variability in care which makes manual procedures less reliable and contributes to a high prevalence of skin diseases.

These problems can be addressed by using automation and remote sensing to prevent conditions leading to skin disease and by avoiding skin disease by directly monitoring skin health and responding to early signs of skin damage, before there are visible manifestations. To accomplish this, a comprehensive understanding of the causes of skin disease and the physiology of the skin would be helpful.

### Skin Structure and pH

Skin has many essential functions. It resists mechanical insults such as tension, torsion, and abrasion; presents a barrier to foreign substances; protects the body from infection; and it regulates water loss. Many skin diseases occur when one or more of these functions are compromised. Many of these functions are influenced by the top layer of the skin known as the stratum corneum. This is a layer of dead skin cells containing mostly the protein keratin, which is also found in the hair and fingernails. This layer varies from 10-50 microns. The stratum corneum provides the skin's barrier function as well as its first line of defense from invasion and mechanical stresses.

Skin health is directly related to the health of the stratum corneum. The integrity of the stratum corneum is highly correlated to its pH which ranges from 4.5 to 6 at the surface (FIG. 1). The pH at the bottom of the stratum corneum is 7. The skin is therefore normally somewhat acidic. This “acid mantle” creates a natural antibacterial effect, especially important in the perianal area where bowel bacteria, such as E. coli, are often present and are the frequent cause of infections. Bacteria from the bowel thrive at a pH of 8 and find the normally acidic skin surface to be inhospitable. Cleansing the skin with a high pH soap or cleanser raises its pH and creates a breeding ground for these bacteria. A higher pH also suppresses the normal skin bacteria whose presence serves to exclude other microorganisms. Studies have shown that hyperacidic treatments can effectively prevent some diseases, such as dermatitis.

The structural integrity of the stratum corneum is also dependent on its pH. The spaces between the cells are filled with lipids, primarily cholesterol, free fatty acids, and ceramides, whose binding strength is pH related. As the pH rises, this binding weakens and the skin becomes more susceptible to abrasion, tension, and shear. Maintaining the skin's normal pH is important for maintaining skin health.

The skin of incontinent people is exposed to urine and feces which are retained by the absorbent products they wear. In the typical institutional setting, a person is exposed to a soiled diaper for 1 hour on average, since checking is performed only every 2 hours. A resident can request assistance if a soiled diaper is sensed, but many residents are either sleeping or mentally compromised and cannot call for help. Even if the resident requests help, none may be forthcoming due to the lack of available caregivers. The prolonged exposure to urine and feces has several effects on the skin. First, there is increased hydration of the skin. Second, there is exposure to high pH material in feces. Third, there is exposure to high pH byproducts of bacterial growth resulting from the mixture of urine and feces. Fourth, increased moisture encourages the growth of microorganisms such as Candida Albicans. Fifth, microorganisms are given more time to spread which can lead to other problems, such as urinary tract infections.

It is commonly assumed that the primary effect of exposure to elevated levels of moisture is to hydrate the skin leading to maceration which then leads to skin disease. Maceration refers to skin changes seen when moisture is trapped against the skin for a prolonged period. The skin turns white or gray, softens and wrinkles. Macerated skin is more permeable and prone to damage from friction and irritants. Maceration leads to changes in the skin that directly affect skin pH which often rises to 7 or 8.

Hence, maintaining the skin surface at the normal pH, or detecting the deviation of the skin pH from its normal levels, would be highly beneficial in preventing skin diseases, particularly in the elderly in healthcare facilities. A surface measurement of pH provides adequate information to deduce the pH gradient through the stratum corneum. Comparison with a baseline reading for each resident can be used to detect changes in pH that indicate skin damage. Treatment to lower pH will help prevent the emergence of skin disease.

### The Causes of Skin Damage

For incontinent residents, prolonged exposure to urine and feces greatly increases the risk of skin disease. It is well known that proper, timely care can prevent skin diseases, but most long term care facilities operate under severe cost and resource constraints that directly affect the quality of care. The resource problem is particularly acute with high levels of turnover, ranging from 40% of Administrators, to 70% of CNAs, and an acute shortage of nurses. The consequence is that residents receive less care than is needed to avoid skin problems.

Care can be directed to the people who need it most by: a) monitoring resident status; b) prioritizing care based on resident specific risks; c) altering priorities in real-time based on resource availability; and d) providing accurate records of care to guide staff training and procedure modifications. There is a serious unmet need for methods and systems that can accomplish the above, cost-effectively.

### Cost Constraints

Any product attempting to address these issues must be low cost. This, of course, means that the unit cost must be low but, more importantly, the labor cost of its use must also be low. The reality of institutional care is that margins are low and costs are rising. Application must be simple, present few opportunities for error, require very little training, and it must be possible to detect misapplication automatically.

Based on the above information, a comprehensive solution incorporating these understandings shall meet the following goals: a) provide a quantitative assessment of skin health to inform skin care procedures; b) provide a timely and accurate notification that a soiled absorbent product needs to be replaced; c) provide remote monitoring; d) require little in the way of caregiver training or effort to apply correctly; d) integrate well with current care procedures to facilitate acceptance; and e) have a cost commensurate with expected savings.

Some of the current solutions that are intended to address the above needs are described below. Many of these are intended to detect a soiled diaper or the need for a change. These include wetness detectors, humidity detectors, and pH indicators.

### Wetness Detectors

The goal of these devices is to detect liquids (e.g., U.S. Pat. No. 7,250,547). These usually rely on conductance changes as measured by conductors placed on a hydrophilic material, such as wires or traces on an absorbent lining (e.g., US Patent Application Publication No. 2005/0033250). They need to be large since they are intended to function when they are in contact with fluids, which may occur over a broad area. Their main problem is false positives. People with bladder incontinence often dribble small amounts of urine more or less continuously. Unless there is some way to detect fluid volume, caregivers will waste time responding unnecessarily. They also have a hard time detecting dry stool. Stools are much more damaging to skin health than urine because of their bacterial load.

### Humidity Detectors

A humidity sensor, monitoring water vapor within the air pocket formed between the skin and the absorbent product, can detect the presence of fluids without being in contact with them. The humidity information can also be used to estimate fluid volume, to reduce false positives. The humidity sensor need not be large, reducing its material costs, and its placement is less critical, reducing opportunities for error and required labor. These have significant advantages over wetness detectors. Existing humidity detectors provide a warning at some set level (e.g., US Patent Application Publication No. 2004/0236302). This is usually set empirically, but is sometimes determined in situ. The humidity within the diaper depends on: a) the water flux from the skin; b) fluids present including from urine and feces; c) the ambient humidity; and d) the rate at which the absorbent product can expel water vapor. There is a change in humidity and temperature when urine is present. An absorbent product with well designed vapor transfer will experience much lower rise in humidity until such time as the absorbent polymer approaches saturation. A useful humidity sensor must consider all these effects to be able to present useful information to the caregiver in a manner that distinguishes between a comfort complaint, a false positive, and a soiled diaper.

### pH Indicators

Several absorbent products have integrated pH indicators. These operate on the principle that the pH of urine rises over time and the rising pH is actually the more important determinant of skin damage than moisture. Some of these solutions provide a visual indication of pH by changing color (e.g., U.S. Pat. No. 4,231,730). Others report pH values for remote monitoring (e.g., U.S. Pat. No. 6,617,488). It is important to note that they measure the pH of urine and not skin pH, and hence provide no indication of the condition of the skin. The pH monitoring of urine and feces suffers from the large variations, 4.6 to 8, due to diet and health. pH sensing provides somewhat better information about soiling, producing somewhat fewer false positives, by using pH buffers to provide some correlation to urine volume. Such solutions have further difficulties when dealing with stools.

### Sensor Integration

All of the solutions above involve sensors integrated into an absorbent product, typically a diaper. There are two basic configurations: a) the sensor and the absorbent product form a unit and are applied and discarded together (e.g., US Patent Application Publication No. 2008/0074274); and b) the sensor comprises two components, a sensing element integrated into the absorbent product, and discarded with it, and a reusable module that interfaces with the sensing element (e.g., US Patent Application Publication No. 2005/0156744). These configurations have three problems: a) they present restrictions on the selection of absorbent products; b) they require special attachments or module replacements during changes, increasing required labor for a change; and c) they discard some components with each change, raising the cost.

Sensors that are integrated into absorbent products pose significant economic challenges to the facilities by increasing total cost. Institutions have different philosophies about absorbent product use: some use cloth diapers, some use super-absorbent disposables. Some use briefs. Some use pads. Some use a combination of different products. Any technology integrated into a specific diaper will either force a change in facility policy or require the manufacturer to offer a bewildering array of product variations. There are a large variety of absorbent products in use. They vary in size, absorbent capability, configuration (e.g., briefs or pants), quality of construction, and disposability, among other characteristics. Of necessity, an integrated sensor and absorbent product can address only a subset of the available options. Hence, a solution that separates the two and leaves the choice of absorbent product independent from the decision to use a sensor would be ideal. Another problem with an integrated solution is inventory control. Most facilities purchase absorbent products in large volumes. Not all users will need to be monitored; perhaps only 50-75% of users, since some are only occasionally incontinent. With a standalone sensor, the choice of who to monitor and when can be made independently of the choice of absorbent product supplier, and adoption of monitoring will not disrupt existing supplier contracts.

The solution involving a reusable component suffers further in that it introduces another step during a change which normally takes 3 minutes. When dealing with an integrated sensor, the Nurse Aide will be required to: a) remove the sensor module; b) clean it; c) reattach the module after applying a fresh diaper; and d) verify it has been done properly. This adds considerably to the cost of a change and presents a major barrier to adoption of the product. The use of connectors to establish electrical contact between the sensor module and sensing element presents a significant risk of application error as well as a source of system unreliability. Connectors are well known system failure points and it is not uncommon to change diapers 10 times a day, a considerable number of connector mating cycles. There have been attempts to get around the connector problem by using inductive coupling techniques (e.g., US Patent Application Publication No. 2004/0036484). All of these techniques still involve a considerable additional effort during each change and represent increased labor cost and reduced efficiency.

An integrated sensing element must be discarded with the absorbent product at each change. This raises the cost dramatically since changes are made 4-10 times per day. A typical diaper might cost $0.35. A sensor element cost (material, assembly, and test) of $0.05 is a substantial percentage cost increase, not including the labor cost of application.

Here it must be repeated that the motivation for remote monitoring of the need to change a soiled diaper is to avoid the costs of treating skin disease. An acceptable product should offer (labor and medical) cost savings to balance the additional sensor product cost. A diaper with an integrated sensor will need to have demonstrable health benefits to attract customers.

There have been some wetness detectors configured as standalone strips to be placed into a diaper during a change. These suffer from being awkward to apply, expensive, uncomfortable, and prone to misapplication. There have also been sensors integrated into bed pads. These have mainly been focused at detecting bedwetting or as aids in continence training.

Wireless vs. Wired

Some solutions involve the generation of local audible or visible alerts (e.g., U.S. Pat. No. 5,264,830). These represent a considerable invasion of privacy in an institutional setting and create a distracting and annoying interruption to caregivers who must respond immediately to silence the alarm, regardless of the relative priority of that resident's needs.

There have been solutions involving a wired connection between a sensor placed into a diaper during a change and a bedside unit. These have been almost universally unsuccessful since they either tether the patient to the bed or fail to operate when the patient is ambulatory. Most solutions rely on wireless techniques to relay sensor readings to some remote unit for display, alerts, or alarms.

Wireless techniques involving radiated RF signals have several problems: a) they consume a lot of power or they require a lot of nearby receiver units to reliably receive low power signals; b) their signals can be easily blocked by nearby objects reducing communications reliability; c) their signals are absorbed or blocked by the body of the wearer; or d) their signals suffer from interference with other RF devices in the environment. Wireless techniques using radiated RF also suffer from privacy issues since the signals are subject to eavesdropping (U.S. Pat. No. 6,603,403).

An animal or human body interferes with most electrical signals. This is a problem when wireless sensors are intended to be placed on or in close proximity to the body but accessed remotely from a significant distance. Increased signal power or antenna orientation restrictions can be employed to overcome this problem, but can create problems of their own effecting ease-of-use or electromagnetic interference or compatibility (EMI/EMC) compliance.

In light of the above, there is a need for a solution which (a) is configured as a standalone sensor usable with any absorbent product; (b) incorporates a humidity sensor for detecting a soiled diaper; (c) incorporates a pH sensor for measuring skin health directly, as opposed to measuring the pH of the excrement; (d) avoids the problems of RF communications; (e) is placed on the skin rather than in a diaper; (f) remains in place through several diaper changes thus reducing daily costs; and (g) integrates information from several sensors to provide more accurate assessments of patient condition. The present invention addresses these and other issues.

### SUMMARY OF THE INVENTION

The present embodiments disclose a wireless sensor system for monitoring local skin condition and using the body as a conduit for signal propagation. In one aspect, the system comprises a wearable network component, as well as one or more wearable sensor components configured to generate a signal indicative of a local skin parameter or an analyte that is in contact with the skin. The local skin parameter may be pH, hydration, conductivity, temperature, or salinity. The analyte may be water vapor, or it may be a compound that is present in feces or urine where the sensor is located. The signal is transmitted from the sensor components to the network component, which in turn relays the signal to one or more external devices via a wireless network. The network component and sensors are configured to communicate by using the body as a conduit for signal propagation by using capacitive coupling between the body and the network component and sensor components.

In one aspect, one or more of the sensor components are configured to simultaneously sense one or more different local skin parameters or analytes in contact with skin. In one aspect, one or more of the sensor components comprise a power source. In another aspect, one or more of the sensor components extract power from their environment or from a signal sent from the network component.

In one aspect, the communication mechanism between the network component and the sensor components comprises a wireless mechanism employing capacitive coupling through a biological medium. In still other aspects, the network component relays requests or commands to the sensor components, for example, to configure or control one or more of the sensor components. In one aspect, the network component is configured to automatically discover the presence and types of sensor components.

In one aspect, the sensor system is used to detect elevated levels of humidity, caused by the presence of human waste materials captured within an absorbent product for managing incontinence, for example, in order to permit the replacement of the absorbent product before skin is damaged due to exposure to body effluents, such as urine and feces. The sensor components may also be used to measure the pH of the skin of the wearer to detect the early onset of skin damage, initiate preventive treatments, and monitor the progress of treatments to ensure rapid restoration of skin health.

These and other embodiments of the present invention will readily occur to those of ordinary skill in the art in view of the disclosures herein.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a graph indicating that the integrity of the stratum corneum is highly correlated to its pH, which ranges from 4.5 to 6 at the surface.

FIG. 2 shows a representative embodiment of the system showing a sensor component and a network component.

FIG. 3 shows a representative embodiment of the system showing several sensor components and one network component.

FIG. 4 shows a representative embodiment of a sensor component.

FIG. 5 is a block diagram of a sensor component.

FIG. 6 shows a representative transmitter circuit.

FIG. 7 shows an example 109 kHz transmitter tank and 2.5 kHz modulation.

FIG. 8 shows tank resonance with stray capacitance.

FIG. 9 shows battery voltage vs. carrier frequency.

FIG. 10 shows a representative embodiment of a network component.

FIG. 11 is a block diagram of a network component.

FIG. 12 shows an example of Differential Manchester encoding.

FIG. 13 is an example coding scheme.

FIG. 14 is an example protocol sequence chart of communications between a network component and two sensor components.

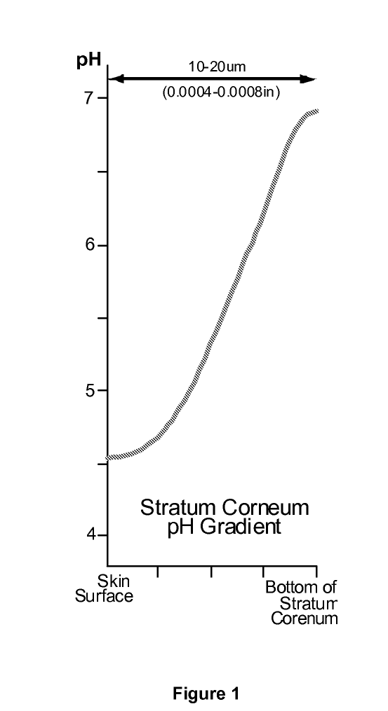

### DETAILED DESCRIPTION

Although the detailed description contains many specifics, these should not be construed as limiting the scope of the invention but merely as illustrating different examples and aspects of the invention. It should be appreciated that the scope of the invention includes other embodiments not discussed in detail above. Various other modifications, changes and variations which will be apparent to those skilled in the art may be made in the arrangement, operation and details of the method and apparatus of the present invention disclosed herein without departing from the spirit and scope of the invention as described here.

### DEFINITIONS

As used herein, the term “local skin parameter” refers to any property of the skin in the vicinity or close proximity of a sensor. Examples of such local skin parameters include, but are not limited to, pH, hydration, conductivity, temperature, or salinity.

As used herein, the terms “analyte” and “target analyte” are used interchangeably and denote any physiological analyte of interest that is a specific substance or component or component mixture that can be detected and/or measured in a chemical, electrical, electrochemical, physical, enzymatic, or optical analysis where a sensor is located. Examples of such analytes include, but are not limited to: water vapor; substances present in body excretions such as feces or urine; substances present in skin excretions such as sweat or excretions of sebaceous glands; chemicals having a physiological action, such as a drug, its metabolite, or pharmacological agent; and the like. A detectable signal (e.g., a chemical signal or electrochemical signal) can be obtained, either directly or indirectly, from such an analyte or derivatives thereof. Furthermore, the terms “analyte” and “substance” are used interchangeably herein, and are intended to have the same meaning, and thus encompass any substance of interest.

As used herein, the terms “biosensor electrode,” “sensing electrode,” or “working electrode” are used interchangeably and refer to an electrode that is monitored to determine the amount of electrical signal at a point in time or over a time period, which signal is then correlated with a measurement of a local skin parameter or the presence or concentration of an analyte. The sensing electrode comprises a reactive surface which converts the skin parameter or analyte, or a derivative thereof, to an electrical signal. The reactive surface may comprise any electrically conductive material such as, but not limited to, platinum-group metals (including platinum, palladium, rhodium, ruthenium, osmium, and iridium), nickel, copper, silver, and carbon, as well as oxides, dioxides, or combinations or alloys thereof.

As used herein, the term “sensor element” or “sensing element” are used interchangeably and refer to, but are not limited to, a biosensor electrode. A sensor element may include one or more components in addition to a biosensor electrode, for example a “reference electrode” and a “counter electrode.” As used herein, “reference electrode” denotes an electrode that provides a reference potential, e.g., a potential can be established between a reference electrode and a working electrode. As used herein, “counter electrode” denotes an electrode in an electrochemical circuit which acts as a current source or sink to complete the electrochemical circuit. Although it is not essential that a counter electrode be employed where a reference electrode is included in the circuit and the electrode is capable of performing the function of a counter electrode, it is preferred to have separate counter and reference electrodes because the reference potential provided by the reference electrode is most stable when it is at equilibrium. If the reference electrode is required to act further as a counter electrode, the current flowing through the reference electrode may disturb this equilibrium. When a reference electrode is required to act further as a counter electrode, a potential drop between the sensor electrode and reference electrode can occur creating perhaps unacceptable errors. Consequently, separate electrodes functioning as counter and reference electrodes are preferred. The terms also refer to a polymer whose electrical characteristics change in the presence of an analyte. This may include the absorption of or reaction with ions or gaseous molecules. The changes in electrical characteristics may include conductance, dielectric strength, or acoustical properties. Sensing of these changes is achieved with one or more electrodes in contact with or close proximity to the polymer or polymers. Polymer chemistry may be selected to react to specific analytes, such as a particular enzyme, or more generally, such as positive ions.

As used herein, the terms “sensor component,” “sensing device,” “sensing mechanism,” or “biosensor device” are used interchangeably and refer to any device that can be used to measure a local skin parameter, or presence or concentration of an analyte or derivative thereof, of interest. Sensing devices comprise, but are not limited to, sensor elements. Sensing devices include electrochemical devices, optical and chemical devices, and combinations thereof. Examples of electrochemical devices include the Kinlen electrode (see U.S. Pat. No. 5,271,820).

As used herein, the term “printed” denotes a substantially uniform deposition of an electrode formulation onto one surface of a substrate (i.e., a base support). It will be appreciated by those or ordinary skill in the art that a variety of techniques may be used to effect substantially uniform deposition of a material onto a substrate, e.g., Gravure-type printing, extrusion coating, screen coating, inkjet printing, spraying, painting, or the like.

As used herein, the term “incontinent product” refers to any of several types of products used to deal with the consequences of incontinence. These include diapers, briefs, pads, and the many variations on these products.

### Overview

The present embodiments disclose a wireless sensor system for using wearable sensor components to monitor local skin conditions. One or more wearable sensor components generate electrical signals indicative of local skin parameters or analytes that are in contact with skin in the vicinity of the sensor components. The generated signals are communicated to a wearable network component. To provide reliable, low cost communication, the body is employed as a conduit for signal propagation between the network component and the sensor components, by using capacitive coupling between the components and the body. The network component relays the signals to one or more external devices.

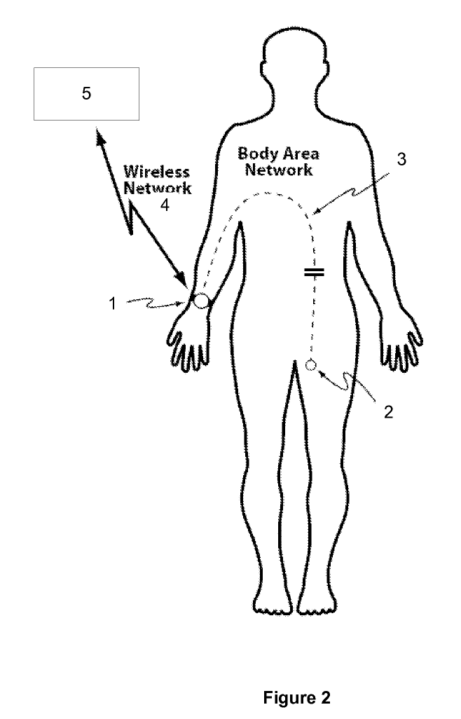

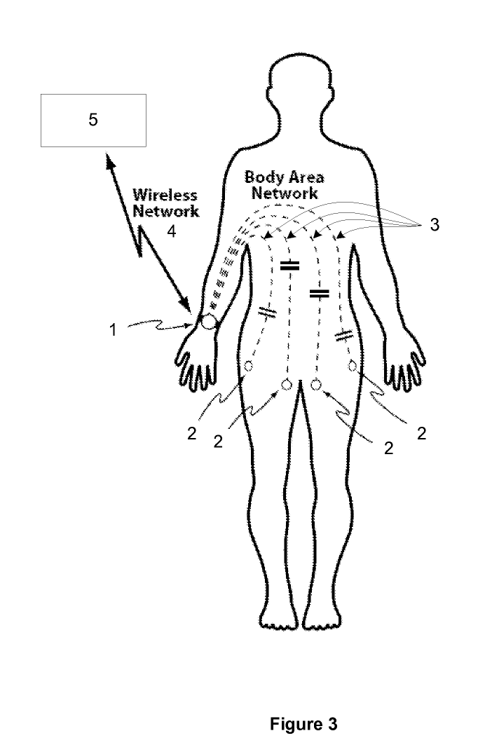

FIG. 2 shows one embodiment of the wireless sensor system comprising a wearable network component 1 and a wearable sensor component 2, which communicate with each other wirelessly. Sensor component 2 communicates with network component 1 via a body pathway 3 by using the body as a signal propagation conduit. Similarly, FIG. 3 shows another embodiment of the wireless sensor system, comprising a plurality of wearable sensor components 2 communicating wirelessly with a wearable network component 1 via body pathways 3.

The sensor components 2 send measurement readings to the network component 1 upon request of the network component 1 and/or periodically. The network component 1 relays the readings over a wireless network 4 to one or more external devices 5 for display, monitoring, analysis, storage, and/or reporting. The network component 1 may also communicate with such devices 5 via a wired network (not shown).

### Sensor Component

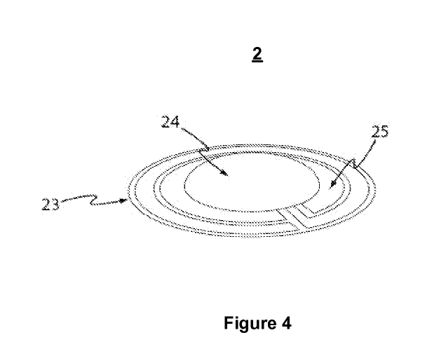

FIG. 4 shows an example embodiment of a sensor component 2. Sensor component 2 comprises a substrate 23 configured to have one or more sensing elements 25 as well as an encapsulated electronics package 24. The sensing element 25 may be affixed on the top and/or to the bottom of the substrate 23. The sensing element 25 and electronics package 24 may be fabricated using techniques that keep the cost very low. Optionally, the sensor component 2 is made of materials that are selected to be disposable.

The sensor component 2 may comprise an adhesive backing to attach to skin. The adhesive backing may be selected to generate an electrolyte with the skin to provide power to operate the sensor component 2. The adhesive may be selected to be compatible with application to an absorbent surface. The sensor component 2 may be flexible so as to be comfortable to the wearer. The substrate 23 may be porous to allow the underlying skin to breathe.

A sensor component 2 detects a physical quantity, representing a local skin condition or analyte in contact with skin, and converts this to a digital value. Optionally, this digital value is combined with a serial number uniquely assigned to each sensor component 2. Optionally, additional status and information bits may be combined into the digital value, as required by the particular application at hand, such as calibration data. The sensor component 2 then encodes this data into a message, for example by pre-pending a preamble and appending error detection and/or error correction bits (such as a checksum). The sensor component 2 then transmits the resulting message to the network component 1 via a body pathway 3. The network component 1 in turn relays the message via a wireless network 3 to one or more devices 5 for display, monitoring, analysis, and storage, and/or reporting.

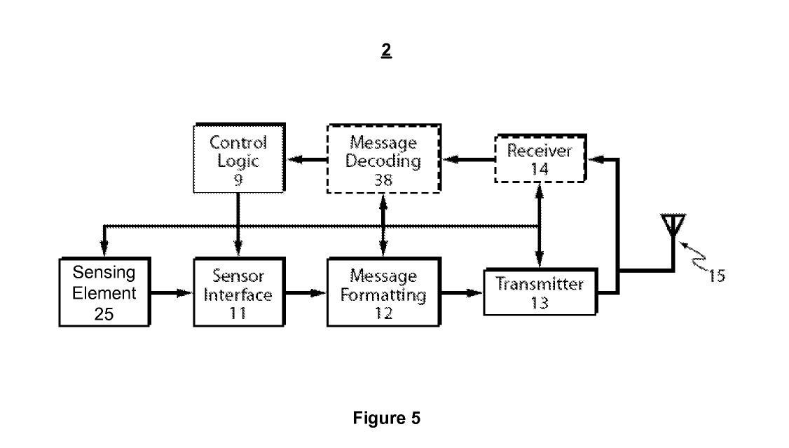

The diagram in FIG. 5 shows the major subsystems of a sensor component 2, in accordance with one embodiment. A sensing element 25 generates a signal indicative of a local skin parameter or an analyte that is in contact with skin. While FIG. 5 shows one sensing element 25, multiple sensing elements 25 are possible, such as individual sensing elements for pH, humidity, temperature, etc. Interface electronics 11 convert the signal generated by the sensing element 25 into a digital form. Interface electronics 11 then optionally combines this sensor reading with other data, such as calibration data, a serial number, a checksum, etc., and encodes it to form a message 12. A transmitter 13 then encodes the message 12 using one of several possible modulation schemes, and sends the message 12 to a receiver 13. The transmission signal uses the body as a conduit for signal propagation, and is coupled to the subject body via an electrode 15. Electrode 15 (also shown below as transmitter plate 44) is in close proximity with the body, but does need to have direct electrical contact with the body.

As described above, a sensor component 2 may optionally be configured to accept signals from a network component 1. This can be accomplished by optionally including a receiver 14 as part of the sensor component 2. The receiver 14 accepts signals from a network component 1 and, after a demodulation and decoding step 38, decodes a message carried by the received signal. Such messages may initiate a measurement reading, request a registration action by the sensor component 2, or assign a sensor address to the sensor component 2. A controller subsystem 9 controls operation of the sensor component subsystems.

Optionally, a sensor component 2 may be configured to: identify itself when queried, for example by broadcasting a hard-coded serial number initialization and waiting for a confirmation response from a receiver; publish its capabilities as part of joining a body area network; or initialize using a broadcast message with retries and random wait until acknowledgment.

Optionally, a sensor component 2 may comprise a power harvesting subsystem or a local power source, such as a battery or a super capacitor.

It is noted that various physical configurations of a sensor component 2 are possible, as long as a transmission element is present to conduct the transmission signal appropriately. For example, in some embodiments, the sensor component 2 is in the shape of a small tab (as shown in FIG. 4). In other embodiments, the sensor component 2 may be a strip, or a tab with wires, or a tab with several extended strips, or integrated into a bandage, or a dressing, or any other configuration suitable for monitoring skin in a desired application.

Optionally, a sensor component 2 is configured to store the measurement of the local skin parameter or analyte for subsequent extraction or transmission.

Optionally, a sensor component 2 may comprises a fractal shape to maximize areal coverage while maintaining inter-electrode separation, impedance, conductance, or capacitance.

Optionally, the signal generated by the sensor component 2 is an electrostatic field conducted through the air with a return path comprising a capacitively coupled electrostatic field conducted through the body.

### Communication Via the Body

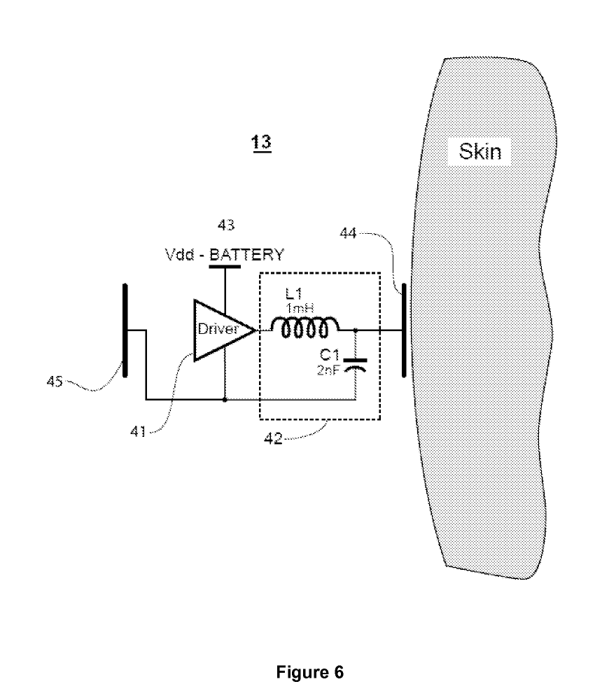

FIG. 6 shows one embodiment of a sensor component transmitter 13. Transmitter 13 comprises a driver 41 and a tank circuit 42 for producing a signal which is capacitively coupled to the body through the skin. The tank circuit 42 runs from a battery 43. In one exemplary embodiment, the voltage of the battery 43 is in the range of approximately 1.5V to 3V, while the output voltage of the transmitter 13 is approximately 20V peak-to-peak or higher. Therefore, tank circuit 42 is configured to be highly resonant, thereby boosting the output voltage and reducing required transmitter 13 current. The tank voltage is applied to two plates 44, 45.

The signal present at the transmitter plate 44 couples to the body and is picked up by the network component 1. A small current, on the order of a few nanoamps, is conducted through the body between these two devices. A return path for this current is provided by the receiving plate 45 which couples capacitively to a similar plate on the network component 1. This return path is normally through the air, but can also be through the ground, if in close proximity.

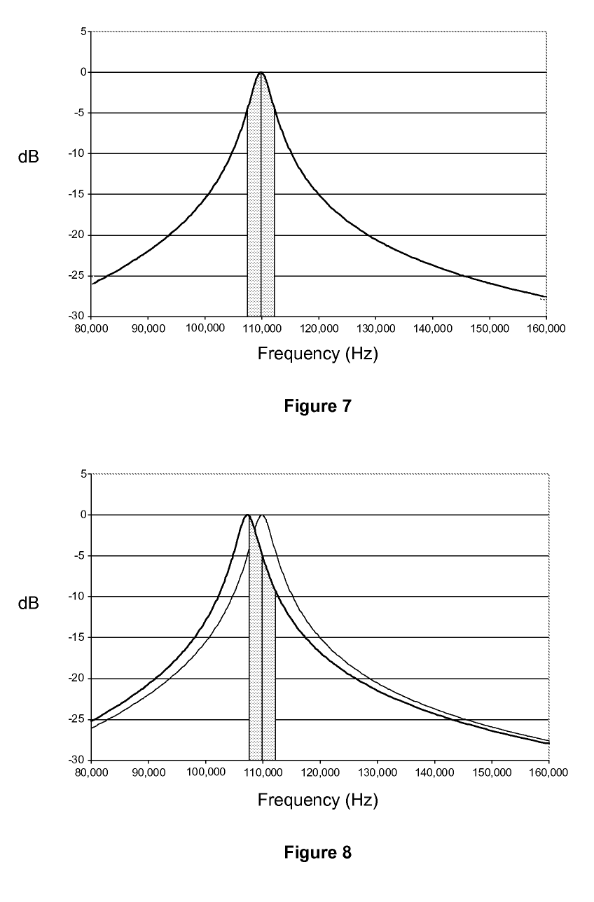

The quality factor (hereinafter also referred to as “Q”) of the series RLC tank circuit is 1/R*SQRT(L/C), where R is the sum of the resistance in the driver and the inductor. A higher inductance leads to higher Q, but constraints of cost and size quickly intervene. In an exemplary embodiment, a combination of a 1 mH inductor with a 2.1 nF capacitor is used for a 109.8 kHz tank resonant frequency. As shown in the frequency plot of FIG. 7, a Q of 30 is achievable. As further shown graphically in FIG. 7, a symmetrical response can be obtained with a ±2.5 kHz modulation at the center frequency.

The tank resonance is sensitive to the values for L and C, which vary due to component tolerance and of which ±20% is typical. An additional capacitive load occurs between the transmitter plate 44 and the skin. The location of the sensor component 2, the condition of the skin, and many other factors may contribute to a significant variation in this capacitance.

It is not unusual to encounter a skin capacitance of 100 pF which, in this example, detunes the tank circuit 42 to approximately 107.3 kHz, as shown graphically in FIG. 8. This small change causes the sidebands of a frequency modulated signal to be mismatched by approximately 9 dB for the 2.5 kHz deviation. This causes distortion in the transmitted signal and reduces the achievable output voltage, thereby making reception and demodulation more difficult. In general, the combined effect of component tolerances and capacitive loading may create considerable uncertainty about the tank resonance, making it desirable to have a compensation scheme.

To keep the modulation balanced and to maximize the transmission signal amplitude, the transmitter carrier frequency can be adjusted to correspond to the actual resonant frequency of the tank circuit 42. This can be achieved by sweeping the carrier frequency and measuring the tank voltage or the corresponding drive current. Both are at a maximum when the carrier is at the tank resonance.

One method for detecting the resonant peak is to monitor the tank voltage directly with a voltage divider feeding an analog-to-digital converter (ADC) or a peak detector. Since the voltage changes are large, the peak is easy to find. This technique requires that the tank voltage be available for monitoring, which may require an additional pin on an application specific integrated circuit (ASIC) which may have no spare pins. Therefore, care is taken to not load the tank circuit 42 significantly.

Alternatively, the resonance of the tank circuit 42 can also be determined by monitoring the tank current. In one embodiment, the tank circuit 42 current is approximately 20 mA at peak. This current can be monitored by inserting a resistor into the tank circuit 42, but since this decreases the tank circuit's 42 Q, the peak is harder to find.

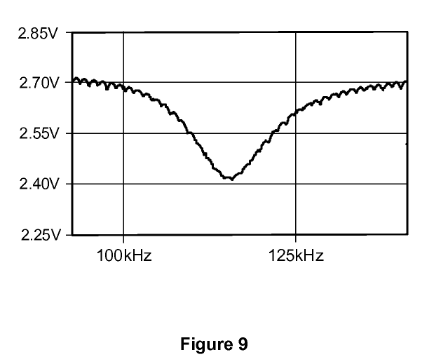

The internal resistance of a typical battery is 10-100 ohms, which can be used to detect the peak. A typical change in battery voltage at the resonance is approximately 20-60 mV. FIG. 9 shows a plot of battery voltage versus carrier frequency, for a representative tank circuit 42. The use of the battery's internal resistance for current monitoring has the advantage of not reducing the tank circuit's Q during the measurement, since no additional resistance has been introduced.

An alternative technique is to measure the current through the output driver to determine tank impedance. A typical complementary metal-oxide semiconductor (CMOS) driver has a low resistance, for example under approximately 10 ohms, but a parallel device can be used with a higher resistance for the purpose of measuring tank current. This has the advantage, over battery voltage monitoring, of using a ground referenced small voltage which can be amplified to produce an easily measured signal.

Once the tank resonance has been determined, the transmission can commence with some assurance that an optimal transmitter performance has been obtained.

Proper selection of the carrier frequency is also important when using other modulation techniques, such as phase or amplitude modulation.

### Sensing Elements

As described above, one or more sensing elements 25 are incorporated into the sensor component 2. These sensor elements 25 detect a local skin condition or analyte in contact with skin and produce a measurable response that is converted into a digital value for transmission. Such sensing elements 25 can be any device including, but not limited to, sensing electrodes, micro-electro-mechanical system (MEMS) devices, semiconductor devices, optical devices, piezoelectric devices, and others.

In one embodiment, a sensor component 2 comprises a sensing element 25 for measuring pH. Such a pH sensing element 25 may be located on the rear surface of the sensor component (i.e., the surface that faces skin) and comprises one or more electrodes designed to measure pH or ion concentration. The pH sensing element 25 may be coated with a polymer gel that serves to provide a medium for ion mobility and to provide adhesion between the skin and the sensor component 2. The pH sensing element 25 may be designed to measure the pH of the top layer of the wearer's skin. This pH should normally be in the range of approximately 4.5-6.

In another embodiment, the sensor component may comprise an additional pH sensing element 25 on the top surface (i.e., the surface that faces away from skin) and used to measure the differential pH between the rear and top surface of the sensor component 2.

In another embodiment, the pH sensing element 25 may comprise two electrodes to form a metal oxide ion sensor. In one embodiment, the sensor component 2 comprises two interdigitated electrodes covered with a polymer treated with an electrolyte. The polymer may be chosen to be sensitive to pH in such a manner that the conductivity of the polymer changes with pH.

Optionally, the pH sensing element 25 may comprise a solid state ion sensor, for example one or more electrodes formed from metals treated to detect ions. Such electrodes may comprise AgCl/AgClO₄ or Cu⁺⁺CuSO₄.

Optionally, the sensing element 25 may comprise a semiconductor device, for example an ion-sensitive field effect transistor (ISFET), which may be integrated into circuitry 24.

In one embodiment, the sensor component 2 comprises a humidity sensing element 25. Such a sensing element 25 measures water vapor concentration in a gas (such as air). In one embodiment, the sensing element 25 comprises interdigitated electrodes covered with a polymer whose conductivity changes with humidity. Optionally, the humidity sensing element 25 is configured to respond only to water vapor and is insensitive to liquids. The humidity level within a diaper provides an accurate indication of the presence and amount of liquid present, even if that liquid has been absorbed within the diaper by some super absorbent polymer. Therefore, the placement of a humidity sensor component 2 is not critical, as long as it is located somewhere within the air pocket created by the diaper and the wearer's body. This makes it possible to place a sensor component 2, configured to have a pH sensing element 25 as well as a humidity sensing element 25, on the wearer's skin at a location chosen to give useful information about skin pH while still being able to sense when the diaper has been soiled.

### Network Component

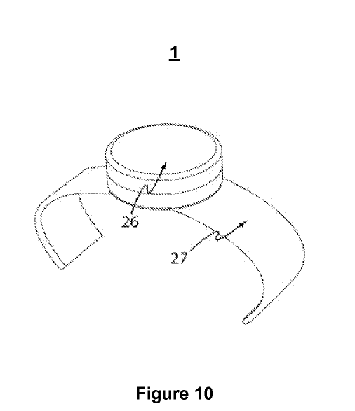

FIG. 10 shows one embodiment of a network component 1. Network component 1 comprises an electronics package 26 (shown in a housing) and an optional strap 27 to attach the network component 1 to the wearer. The network component 1 may also be incorporated into a belt, armband, ankle bracelet, necklace, headband, or any other article that can be worn in close contact with the subject. A network component 1 may also be placed into a pocket or integrated into a chair, bed, walker, or other equipment or accessory.

Since some patients may object to wearing a network component 1, the network component 1 can easily be designed into a bed pad to receive a sensor component's 2 signals from the patient. This flexibility is a key advantage of the present system architecture. Therefore, as should be obvious to one of ordinary skill in the art, the present system architecture allows many possible configurations, and the description herein is not to be interpreted as a comprehensive summary of all such configurations.

The network component 1 comprises a power source, such as a battery. The power source may be rechargeable, in which case the network component 1 is configured to allow for recharging the power source. In some embodiments, power may be extracted from the environment using a parasitic power subsystem.

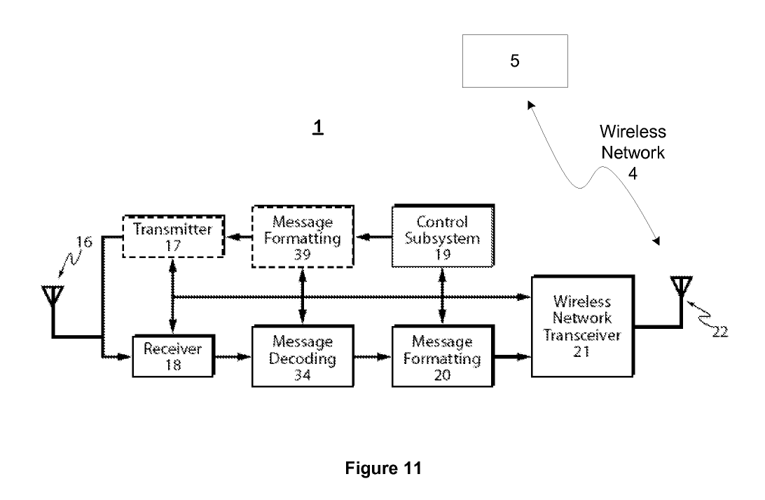

FIG. 11 shows the major subsystems of the network component 1. Signals from sensor components 2 are received at an electrode 16. The received signal is demodulated by a receiver 18, and the signal's message is decoded by a message decoding subsystem 34. The decoded message is then formatted, by a message formatting subsystem 20, into a message for broadcast by a network transceiver 21, 22 over a network 4 to one or more external devices 5, as described above. These subsystems are controlled by a processor or logic system 19.

As described above, a network component 1 may optionally be configured to send messages to sensor components 2 to initiate data requests from sensor components 2, discover new sensor components 2, or register new sensor components 2. This can be accomplished by optionally including a transmitter 17 as part of the network component 1. The network component 1 may transmit a message to explicitly request data from one or more sensor components 2, or it may wait for sensor components 2 to transmit data independently. In the latter case, the network component 1 is configured with a “wake on receive” mode that uses energy from the beginning of the sensor component message to wake the network component 1 from its sleep mode so that the network component 1 can receive the message.

Optionally, a network component 1 may be configured to: identify itself when queried, for example by broadcasting a hard-coded serial number initialization and waiting for a confirmation response from a receiver; publish its capabilities as part of joining a body area network or a wireless network; or initialize using a broadcast message with retries and random wait until acknowledgement.

The network component 1 may be configured to communicate using any desired wireless network, or using one or more wireless networks. In one embodiment, the ZigBee (IEEE 802.15.4) low power standard is used. WiFi (IEEE 802.11) and Bluetooth are alternative wireless technologies that can be used. Optionally, the network component 1 may be configured to communicate with a mesh network, a Transmission Control Protocol/Internet Protocol (TCP/IP) network, an Ultra Wide Band (UWB) network, or a GSM/CDMA network.

In one embodiment, the network component 1 is configured to locally store data received from one or more sensor components 2 until such time as network communication is reestablished or until the network component 1 is prompted to send its data to an external device 5.

Optionally, the network component 1 is hermetically sealed to prevent contamination or damage.

Optionally, the network component 1 may contain sensors to evaluate the local conditions and supplement the information obtained from the sensor component 2. In one embodiment, the network component contains a humidity sensor which can be used to determine the ambient humidity to improve the ability to assess the humidity value from the sensor component 2.

Communication between Components

As described above, in one embodiment a sensor component 2 transmits its data to a network component 1 using the wearer's body itself as a signal propagation conduit. This avoids the shadowing and absorption problems faced when attempting to send electromagnetic signals by radiation. Since the wearer may have a sensor component 2 between the legs, or may be sitting on a sensor component 2 while in a wheel chair, no clear transmission path can be relied upon.

A sensor component 2 generates a signal which is coupled to the body capacitively. As described above and shown in FIG. 6, this can be done in an LC tank circuit tuned to a carrier frequency which can be any low-frequency signal, for example in the range of approximately 30 kHz to 300 kHz. The signal is modulated with digital data that encodes the sensor readings and other information. Several modulation schemes are possible, including but not limited to frequency shift keying (FSK), phase shift keying (PSK), frequency modulation (FM), phase modulation (PM), amplitude modulation (AM), spread spectrum, etc. Several data encoding schemes are possible including RZ, NRZ, Manchester, etc.

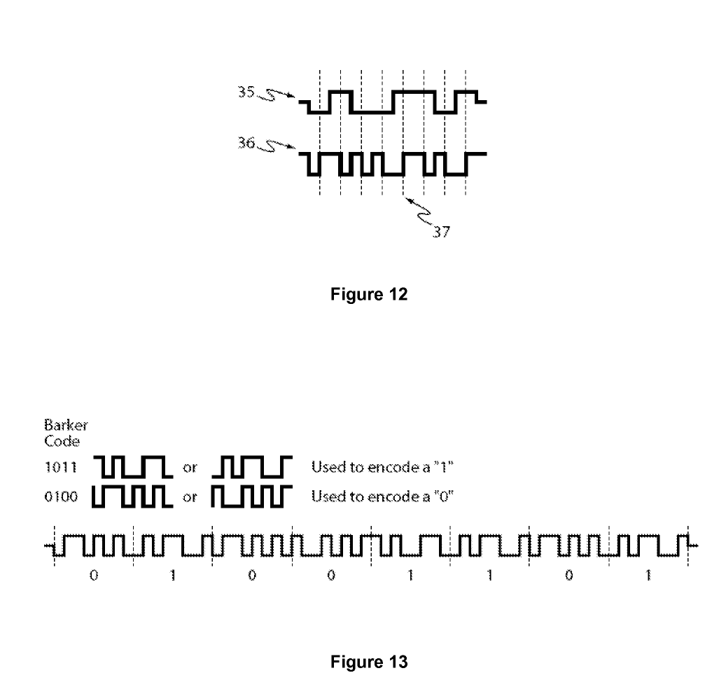

In one embodiment, the encoding scheme is Differential Manchester. This encoding is shown in FIG. 12, which shows an example data stream 35 and the corresponding Differential Manchester encoding 36. Note that there are transitions in the middle of a bit period 37. A “0” has an additional transition at the start of the bit period. This encoding is also used in disk drives for the drive's self clocking capability.

A further encoding of the message is desirable to prevent or inhibit interference of extraneous noise with the signal. In one embodiment, a Barker code may be used to mark the beginning and ending of a message. Barker codes can be used to represent the data itself. One such encoding is shown in FIG. 13. Other coding schemes, such as Walsh codes, are also possible. The message may include a checksum, cyclic redundancy check (CRC), or some other mechanism to allow the network component 1 to verify the accuracy of the transmission.

In one embodiment, the network component 1 may receive the sensor component 2 signal using a code locked loop, spread spectrum, or other technique designed to improve signal-to-noise ratio (SNR) and reliability of communication.

In one embodiment, a sensor component 2 sends its message unilaterally, expecting network component 1 to be ready to receive it. In another embodiment, the network component 1 sends a signal requesting a sensor component 2 to take a measurement reading and respond with the data. The network component 1 may be configured such that when it receives a message in error, it may request a re-transmission.

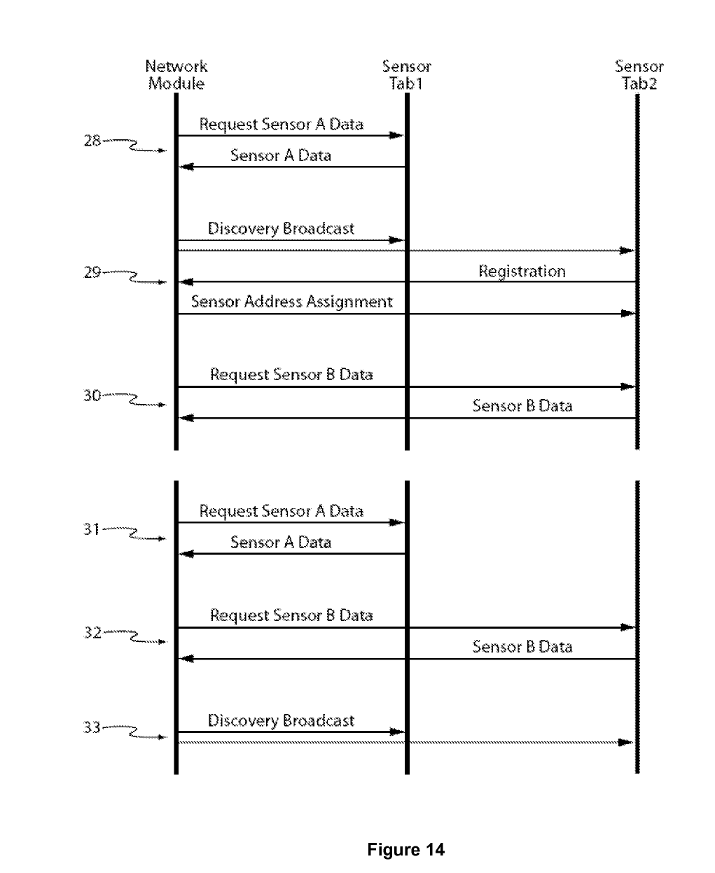

In one embodiment, a network component 1 automatically discovers the sensor components 2 that are present. Such an operational scheme is depicted in the diagram of FIG. 14. The network component 1 broadcasts a signal 28 that asks a specific sensor component 2 for a reading. If that sensor component 2 is still present, it sends a response. This process is repeated for several (or all) sensor components 2 registered with the network component 1. After readings have been collected from all sensor components 2, network component 1 broadcasts a discovery message 29 asking any unregistered sensor components 2 to respond. The sensor components 2 respond with registration messages at random intervals. The network component 1 receives these messages and, after pre-defined maximum interval has passed, sends an address assignment message back to each sensor component 2 that registered. This address will be used from that point on by the network component 1 to request sensor readings. In one embodiment, if any sensor registration messages collide, the network component 1 ignores them and sends another discovery broadcast message. This process is repeated until no more sensor components 2 respond to discovery messages. Once the new sensor components 2 have been registered, the network component 1 may proceed with requesting sensor data from the newly registered sensor components 2 via request message 30.

When no new sensor components 2 are present, the network component 1 asks for readings from the registered sensor components 2 via messages 31 and 32 and, receiving no response from the discovery message 33, may start a waiting period for the next sensor data gathering. Sensor components 2 that no longer respond to sensor data requests may be assumed to have been removed from the wearer. This information is reported to caregivers so that a non-functioning sensor component can be replaced (if it has not in fact been removed from the wearer).

In an alternative embodiment, the network component's 1 receiver 18 is enabled to receive transmissions from sensor components 2 at any time. When a signal is received, the network component 1 may wake up from a sleep state to process the transmission.

In a further embodiment, the sensor components 2 transmit only after they detect a beacon signal from a network component 1. Upon receipt of the beacon, the sensor components 2 transmit their data, delayed by a random time period. The seed for this random delay may be extracted from a sensor component's 2 serial number. The random delays serve to separate the transmissions of several sensor components 2 to reduce the probability that their transmissions overlap. The network component 1 may detect when a message is corrupted by transmission collision and retransmit the beacon signal.

In yet a further embodiment, the sensor components 2 may listen for an existing transmission signal before transmitting. This will further reduce the chance for collisions.

Optionally, a network component 1 is configured to: maintain contact with sensor components 2 and report failure; detect when a sensor component 2 is removed or replaced; detect a presence of a new sensor component 2; generate an alert message based on a manually activated event; generate multiple priority level messages when communicating with the wireless network; dynamically route messages when communicating with the wireless network; or sense other network components in a path of the signal and to communicate directly with them.

In alternative embodiments, communication between the network component and sensor components may comprise a wireless mechanism employing electromagnetic waves; capacitive coupling through a biological medium; inductive coupling; infrared coupling; or a combination thereof.

### Power for Components

In one embodiment, a sensor component 2 includes a power source which comprises a battery, a super capacitor, or similar. The power requirements of a sensor component 2 are low enough that thin film batteries are practical. As an example, a typical sensor component may require less than 0.3 mAH to operate for a day.

In one embodiment, a sensor component 2 extracts power from the environment. Such techniques include harvesting energy from vibrations, harvesting thermal energy, harvesting energy from light, or harvesting energy from RF signals. In one embodiment, an electrolyte gel forms a low power battery when applied to the body. The current from this battery is collected by the sensor component's 2 integrated circuit (IC) and used to charge a capacitor. The IC operates from that capacitor's charge when taking sensor readings and transmitting the results.

A sensor component 2 may include a mechanism for charging its internal power source using, for example, inductive coupling. This can be used to charge a battery or super capacitor just before application. The sensor component 2 may also comprise a power monitoring mechanism for reporting on the status of the power source.

The network component 1 may comprise a battery and a recharging system, such as an inductively coupled charger. In one embodiment, a power harvesting mechanism is included to extract power from mechanical motion of the network component or from electrical signals present in the environment. One such mechanism uses a tuned coupling with a power source to extract power at some distance. The network component 1 may be configured to monitor its internal power source and communicate on its power status via the network 4 in order to prevent unexpected power loss.

### EXAMPLES

There are many applications of the wireless sensor system described herein. In one application, the sensor components 2 are placed on the skin or in the diaper of an incontinent patient during a diaper change to monitor the pH of the patient's skin to assist in care giving activities. In this application, control of skin pH will lead to a reduction in skin diseases, including dermatitis and decubitus ulcers.

In another application and as described above, a sensor component 2 for sensing humidity is used to detect when a diaper has been soiled. Humidity is a more reliable indicator of soiling than is moisture, since a humidity sensor component 2 measures water vapor which readily moves throughout the interior spaces in a diaper. In contrast, a moisture sensor must be present where liquids appear, or have a mechanism for wicking liquids to the sensor, in order to reliably detect soiling. Tests show that the humidity in the interior spaces of a diaper reliably indicates the presence of liquids only a short period of time following soiling. The humidity level reflects the quantity of liquid present, thus avoiding false positive alerts.

It is an advantageous aspect the that present embodiments provide patient-specific information about when to change diapers to avoid high moisture and pH levels, but not to change diapers unnecessarily. They also provide a quantitative assessment of skin health to support preventative treatments to avoid skin disease. Skin diseases can be prevented by eliminating prolonged exposure to urine and feces which cause rising skin pH, maceration, increased permeability, reduced resistance to abrasion, and other effects. The annual savings in labor and materials can be significant, or staff can be freed for other priorities within the facility, while at the same time improving the resident's quality of life by maintaining skin health. Managing skin health by monitoring and controlling skin pH and moisture can reduce the incidence of dermatitis, rashes, skin tears, and the prevalence of pressure ulcers (decubitus ulcers).

It is another advantageous aspect that the present embodiments provide methods for assessing the actual health of the person being monitored to: a) prioritize caregiver resources; b) detect early onset of skin damage to initiate mitigation procedures; and c) monitor the progress of treatments intended to restore skin health. Resource scheduling realities make it plain that immediate response to an alert will not always occur. The invention adjusts priorities for care based on a real-time monitoring of the care provided, the assessed risk of skin disease, and the causal history for each resident. This information permits allocation of resources where they're most needed.

In another application, a sensor component 2 is integrated into a dressing or bandage to be placed on a wound. The sensor component 2 monitors the healing process using one or more sensing elements 25 including, for example, pH and moisture sensing elements 25.

In still another application, a sensor component 2 is used in monitoring healing on a wound underneath a cast.

In still another application, a sensor component 2 is used to monitor the anesthesia during surgical procedures by monitoring skin conductance, sweat, and/or salinity.

In still another application, a sensor component 2 is integrated into clothing to monitor pressure points in the interest of avoiding undue pressure at sensitive locations. This is useful for avoiding pressure related skin breakdown leading to decubitus ulcers.

In still another application, a sensor component 2 is integrated into a tab, the sensor component 2 comprising sensing elements 25 for monitoring respiration and pulse for continuous patient monitoring or for monitoring stress during activities.

In still another application, a sensor component 2 is used for monitoring the skin health of infants, whose acid mantle doesn't mature until two weeks after birth, and others at risk of skin damage, such as workers exposed to hazardous environments. Routine assessments of skin pH can be used to assess general skin health as part of a skin care regimen.

In still other applications, the present embodiments may be used not just with humans, but also with other animals.

### Skin Disease Prevention

In one embodiment, sensor components 2 are used to prevent skin disease and to monitor and assess skin disease treatment. People with incontinence are at elevated risk of skin disease. This is due to the damage to their skin caused by prolonged exposure to excessive levels of moisture and the chemical effects of exposure to human waste products.

Excessive levels of moisture can occur because of the presence of urine or feces. Excessive levels can also occur due to the entrapment of moisture naturally released through the skin and trapped inside an incontinent product (such as a diaper). There are many examples of the use of liquid sensors intended to detect the presence of urine. However, these often fail for two reasons: first, the sensors need to be in contact with the liquids for detection to occur, making sensor placement and configuration very important; and second, it is difficult to measure liquid quantity with such sensors, leading to false positives.

These and other problems may be avoided by the use of a sensor component 2 for sending humidity. The humidity within an incontinent product rises rapidly in the presence of liquid, even though the liquid is being absorbed by the product (into a polymer, for example). Tests have shown that the humidity level throughout the interior spaces of a diaper quickly rises when liquids are present. Furthermore, the humidity level provides a good estimate of liquid volume. A humidity sensor component 2 as described here can therefore be placed in any convenient location and still be capable of detecting liquids. Elevated levels of humidity will be present in the case of excessive water loss by the wearer (for example from sweat), which can also lead to skin damage.

Skin health is closely correlated to skin pH. Studies over the past 10 years have discovered the underlying mechanisms and reinforced the central role pH plays in skin health. A sensor component 2 for sensing pH, placed on the skin, can provide important information about skin health by providing quantitative assessment of pH and changes in pH. This information can be used to anticipate skin damage and to take preventive steps. The pH readings can also be used to monitor skin treatments for efficacy.

Today, skin disease is detected visually, by noting the presence of skin damage. Thus, skin treatment is reactive. The present embodiments allow the use of pH in proactive procedures that can prevent skin disease from occurring.

In one embodiment, one or more sensor components 2 are used for the prevention of skin disease, as follows: One or more sensor components 2 for sensing pH and humidity are placed on the skin of the incontinent person at each change of an incontinent product. The sensor components 2 are placed in one of several critical locations where skin damage is most likely to occur. Should a skin pH measurement not be needed, the sensor components 2 can be placed on the inside surface of the incontinent product. The system periodically monitors and reports humidity and pH levels. The length of time between readings may be adjusted based on the risk factors of each wearer. When the humidity levels exceed a calculated threshold, an alert is generated informing caregivers that the incontinence product is due to be changed. This prevents prolonged exposure that can lead to skin damage. The status of each wearer is displayed by the system with an attendant risk assessment determined by a combination of: a) local policy and procedures; b) ambient comparison data; c) historical wearer-specific sensor readings; d) assessed wearer-specific risk; e) historical patterns of care (including typical response time); and f) data linking health outcomes (skin disease) to actual care provided (timeliness vs. sensor readings). A standard model can be used by default. The model parameters can be modified for wearers with increased risk of skin disease. A record of the humidity and pH levels may be retained. From this information, a risk profile for the wearer may be extracted in order to modify the risk assessment (usually, this will increase the estimated risk level). This information may also be used to assess staff response time to the need for changing an incontinent product. When an elevated skin pH is detected, treatment may begin in order to bring the pH level back down. Normal skin pH is in the range of approximately 4.5-5.5. During the course of treatment, pH readings by the sensor components 2 may be used to assess effectiveness of treatment. If the pH level does not improve, a change in treatment protocol may be needed. Sensor components 2 with more than one pH sensing element 25 may be used to provide additional information about exposure levels and risk assessment. For example, if the urine is very alkaline, a change in diet may be appropriate for high risk wearers.

Using the sensor components 2 as described above, the incidence of skin diseases can be greatly reduced. Monitoring treatments as described leads to reduced prevalence as well. Improving skin health, through the monitoring and control of skin pH, can reduce diseases caused by weakened skin. Among these are decubitus ulcers, dermatitis (in several forms), skin tears, shears, rashes, and others.

Optionally, based on gathered information about skin condition, one or more chemical or non-chemical skin treatments may be administered. For example, the skin may be treated with chemical compositions such as lotions, creams, gels, tonics, sticks, sprays, ointments, pastes, powders, mousse, shampoos, conditioners, oils, colorants, and biomedical and dermatological treatments.

### Other Functions of the Network Component

The network component's 1 main purpose is to communicate with the sensor components 2 and relay their readings over a network to one or more devices for display, monitoring, analysis, storage, and/or reporting. However, the network component 1 may optionally provide other functions as well, as will be presently described.

In one embodiment, a network component 1 comprises one or more accelerometers configured for monitoring patient orientation, activity, and/or falls. For example, when the network component 1 is located at the waist of the wearer, accelerometers detecting orientation and movement can provide sufficient information to allow caregivers to avoid unneeded re-positioning of the wearer, thus avoiding sleep disruptions at night.

In another embodiment, a network component 1 comprises a mechanism whereby the location of the network component 1 may be determined or estimated through interactions between the network component 1 and other devices on the network with which the network component 1 is communicating. This allows the network component 1 to send information about the location of the wearer, thereby allowing tracking of the wearer. Optionally, the network component 1 may comprise a location detection element, such as a Global Positioning System (GPS) element, for tracking the wearer.

In still another embodiment, a network component 1 comprises one or more sensor components 2 of its own, such as sensor components 2 for sensing temperature, respiration, acceleration, vibration, humidity, light, sound, or vital signs. The network component 1 may then combine such information with data obtained from other sensor components 2 for relaying over the network, or the network component 1 may alternatively send out such information independently.

### Privacy

Since communications between sensor components 2 and network component 1 pass through the subject's body, it is very difficult for a 3rd party to intercept the data. This helps protect the patient's privacy. To enhance this, the protocol between the network component 1 and sensor components 2 may include a scheme that prevents other network components from detecting and communicating with sensor components present on another subject. The potential for a network component to detect another subject's sensor components may occur when two subjects come into contact, such as when shaking hands. To address this, a sensor component 2, once registered, may be configured to only communicate with the network component 1 with which it has registered.

In one embodiment of the system, an exception may be provided to allow certain, specially configured network components 1 to communicate with sensor components 2 that are not registered with the network components 1. This allows a nurse or doctor, for example, to obtain real time readings from sensor components 2 by using a specially configured network component 1 which they bring into contact with the subject. An example of such a configuration is a network component 1, optionally comprising a display, wherein the network component 1 is configured to poll sensor components 2 and display the results in order to give the caregiver an immediate confirmation of the sensor component 2 readings, which readings are also presented via the network in the normal manner described above.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A wireless sensor system using the body as a conduit for signal propagation, comprising:
a wearable network component configured to use the body as a conduit for signal reception by using capacitive coupling between the network component and the body, the network component configured to receive a signal from a wearable sensor component, wherein the signal is indicative of a local skin parameter or an analyte in contact with skin.

2. The sensor system of claim 1, wherein the signal is indicative of the local skin parameter, and wherein the local skin parameter is pH, hydration, conductivity, temperature, or salinity.

3. The sensor system of claim 1, wherein the signal is indicative of an analyte in contact with skin, and wherein the analyte is water vapor or is present in feces or urine where the sensor component is located.

4. The sensor system of claim 1, wherein the network component is further configured to transmit the signal to an external device through a wireless network.

5. The sensor system of claim 1, further comprising:
at least one wearable sensor component configured to be in contact with skin, wherein the sensor component generates the signal and transmits the signal to the network component by using capacitive coupling between the sensor component and the body.

6. The sensor system of claim 5, wherein the sensor component is configured to simultaneously sense two or more different local skin parameters or analytes in contact with skin.

7. The sensor system of claim 5, wherein the sensor component comprises a power source.

8. The sensor system of claim 5, wherein the sensor component is configured to extract power from its environment.

9. The sensor system of claim 5, the sensor component comprising:
a sensing element; and
circuitry for generating the signal and transmitting the signal to the network component.

10. The sensor system of claim 5, further comprising:
a device configured to receive the signal from the network component and to display, monitor, analyze, store, or report the signal.

11. A sensor device configured to be in contact with skin and uses the body as a conduit for signal propagation, comprising:
a sensing element configured to sense a local skin parameter or an analyte in contact with skin; and
circuitry for generating a signal indicative of the local skin parameter or the analyte that is in contact with skin, and for transmitting the signal to a network component by using capacitive coupling between the sensor device and the body.

12. The sensor device of claim 11, wherein the signal is indicative of the local skin parameter, and wherein the local skin parameter is pH, hydration, conductivity, temperature, or salinity.

13. The sensor device of claim 11, wherein the signal is indicative of an analyte in contact with skin, and wherein the analyte is water vapor or is present in feces or urine where the sensor device is located.

14. The sensor device of claim 11, wherein the sensor device is configured to simultaneously sense two or more local skin parameters or analytes in contact with skin.

15. The sensor device of claim 11, further comprising a power source.

16. The sensor device of claim 11, wherein the sensor device is configured to store the sensed parameter or analyte for subsequent extraction or transmission.

17. A method of monitoring local skin condition using the body as a conduit for signal propagation, comprising:
generating a signal indicative of a local skin parameter or an analyte that is in contact with skin, wherein the generating is accomplished using a wearable sensor component; and
transmitting the signal from the wearable sensor component to a wearable network component by using capacitive coupling between the sensor component and the body.

18. The method of claim 17, wherein the signal is indicative of the local skin parameter, and wherein the local skin parameter is pH, hydration, conductivity, temperature, or salinity.

19. The method of claim 17, wherein the signal is indicative of an analyte in contact with skin, and wherein the analyte is water vapor or is present in feces or urine where the sensor component is located.

20. The method of claim 17, further comprising:
relaying the signal from the network component to an external device through a wireless network.

21. The method of claim 17, wherein the signal is indicative of two or more different local skin parameters or analytes in contact with skin.

22. The method of claim 17, further comprising:
extracting power from the sensor component's environment, or from a signal sent from the network component, to power the sensor component.
